(19) United States
(12) Patent Application Publication
(10) Pub. No.: US 2024/0261494 A1
(43) Pub. Date: Aug. 8, 2024

(54) SHOWERHEAD, SINK FAUCET ENEMA, AND VAGINAL WASH HYGIENE ATTACHMENT KIT

(71) Applicant: SUNDAY OKPALA-EZECHUKWU, Cochrane (CA)

(72) Inventor: SUNDAY OKPALA-EZECHUKWU, Cochrane (CA)

(21) Appl. No.: 18/632,218

(22) Filed: Apr. 10, 2024

(51) Int. Cl. A61M 3/02

(52) U.S. Cl. CPC A61M 3/0279; A61M 3/022; A61M 2205/7545; A61M 2205/759

(57) ABSTRACT

The present system introduces a showerhead and sinks faucet kit in personal hygiene technology, offering a comprehensive solution for enema and vaginal wash hygiene practices. A crafted from medical-grade, BPA-free materials, this innovative showerhead and sink faucet attachment kit comprises a range of components designed to ensure convenience, hygiene, and user-friendliness. From different nozzle tip attachments catering to individual preferences to a flexible silicone tube-hose facilitating water transport, each part is meticulously engineered to provide optimal performance and comfort. The water flow pressure regulator switch valve and secure threaded coupling connector fittings, users can customize their hygiene experience while maintaining safety and reliability. The water filtration mesh washers and lock-in washers further enhances hygiene standards, ensuring clean and continuous water flow during procedures. Versatile and adaptable, the kit's stretchable universal water inlet rubber silicone attachment ensures compatibility with various water sources, offering convenience for users across different settings.

### TECHNICAL FIELD

The present invention relates to the field of enema kit, and more particularly to a nozzle assembly connected an enema kit for supplying water.

### BACKGROUND

The following description includes information that may be useful in understanding the present invention. It is not an admission that any of the information provided herein is prior art or relevant to the presently claimed invention, or that any publication specifically or implicitly referenced is prior art.

Traditional approaches to enema and vaginal wash hygiene have historically relied on manual equipment and techniques. While these methods have been effective to some extent, they often come with inherent challenges and drawbacks. Manual filling of enema or douche devices with water or solutions can be cumbersome and time-consuming, discouraging consistent adherence to hygiene practices. Moreover, ensuring adequate hygiene and safety standards with traditional equipment presents ongoing challenges, particularly concerning proper sterilization procedures and prevention of contamination.

One of the critical limitations of conventional enema and vaginal wash methods lies in the lack of a continuous and ample water supply during procedures. This issue can lead to interruptions and inconvenience during the hygiene process, potentially compromising its effectiveness. Additionally, the availability of specialized equipment for enema and vaginal wash procedures may be limited, particularly in underserved communities or regions with restricted access to healthcare resources.

Manual filling of enema or vaginal wash devices requires additional time and effort, potentially deterring individuals from incorporating regular hygiene practices into their routines. Further, ensuring proper sterilization and preventing contamination with traditional equipment can be challenging, raising concerns about hygiene and safety during hygiene procedures. The availability of a continuous and sufficient water supply is essential for effective enema and vaginal wash procedures. However, traditional methods may suffer from limited water capacity, leading to interruptions and suboptimal hygiene outcomes.

Access to specialized enema and vaginal wash equipment may be restricted, particularly in rural or low-income areas. This lack of accessibility can hinder individuals' ability to maintain proper hygiene and may contribute to health disparities.

In light of these challenges and drawbacks, there exists a compelling opportunity for innovative solutions that address the limitations of traditional enema and vaginal wash methods. As a result, there exists a need for improvements over the prior art and more particularly for a more efficient way.

### SUMMARY OF THE INVENTION

In addressing the limitations of traditional enema and vaginal wash methods, the present invention offers unparalleled convenience and accessibility. By providing a continuous and ample water supply during hygiene procedures, it eliminates interruptions and enhances the effectiveness of cleansing. Moreover, its user-friendly design and universal compatibility make it suitable for a wide range of users, including those with limited mobility or access to healthcare resources.

The invention revolutionizes enema and vaginal wash hygiene practices. Designed with convenience, hygiene, and user-friendliness in mind, this innovative attachment kit seamlessly integrates with standard showerheads and sink faucet, providing users with a continuous and ample supply of water during hygiene procedures. The Showerhead, Sink Faucet Enema, And Vaginal Wash Hygiene Attachment Kit comprises a medical-grade, BPA-free clear silicone rubber components, ensuring safety, durability, and ease of maintenance. It's universal attachment mechanism allows for effortless installation onto most showerheads and sink faucet, eliminating the need for specialized plumbing or tools. Users can simply attach the showerhead and Faucet kit to their preferred water source and adjust the flow rate and pressure to suit their individual preferences and comfort levels.

One of the key features of the hygiene attachment kit is its versatility. It comes equipped with a range of nozzle attachments designed specifically for enema and vaginal wash purposes. These nozzles are ergonomically designed and come in various shapes and sizes to accommodate different anatomies and user preferences. Additionally, the kit includes controls for adjusting water flow and temperature, ensuring a comfortable and personalized experience for users. Hygiene and safety are paramount considerations in the design of the Kit. The medical-grade silicone rubber material is resistant to bacterial growth and easy to clean, maintaining optimal hygiene standards with minimal effort. Furthermore, the showerheads and sink faucet attachment kit incorporates backflow prevention mechanisms to prevent contamination of the plumbing system, ensuring safe and hygienic operation.

This showerheads and sink faucet attachment kit includes a variety of BPA-free nozzle tip attachments, each catering to different anatomical needs and user preferences. A water flow pressure regulator switch valve provides users with precise control over the intensity of the water flow, ensuring a customized and comfortable cleansing experience. The showerheads and sink faucet attachment kit further incorporates a stretchable universal showerhead and sink faucet water inlet rubber silicone attachment, ensuring compatibility with a wide range of water sources for enhanced convenience.

Furthermore, essential components such as the nozzle tips, switch valve, and connectors are made from BPA-free food-grade PVC material, prioritizing safety and hygiene standards. Additionally, a flexible silicone tube-hose facilitates the transportation of cleaning water between components, ensuring smooth and efficient operation throughout the procedure. By combining versatility, comfort, and safety, this hygiene attachment kit sets a new standard for hygiene procedures, offering users a seamless and hygienic cleansing experience.

The summary of the invention does not necessarily disclose all the features essential for defining the invention. The invention may reside in a sub-combination of the disclosed features. The various combinations and sub-combination are fully described in the detailed description.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

The diagrams are for illustration only, which thus is not a limitation of the present disclosure, and wherein:

FIG. 1 illustrates a showerhead and sink faucet attachment kit for enema and vaginal wash hygiene, in accordance with a preferred embodiment of the invention.

FIG. 2 illustrates various elements of the showerheads and sink faucet attachment for enema and vaginal wash hygiene, in accordance with a preferred embodiment of the invention.

FIG. 3 illustrates connection and filtration part of the showerheads and sink faucet attachment for enema and vaginal wash hygiene, in accordance with a preferred embodiment of the invention.

FIG. 4 illustrates exploded view of the connection and filtration part of the showerheads and sink faucet attachment for enema and vaginal wash hygiene, in accordance with a preferred embodiment of the invention.

FIG. 5 illustrates view of the female top PVC threaded coupling connector fitting, in accordance with a preferred embodiment of the invention.

FIG. 6 illustrates view of the male bottom PVC threaded coupling connector fitting, in accordance with a preferred embodiment of the invention.

FIG. 7 illustrates view of the Water Filtration Mesh Washer, in accordance with a preferred embodiment of the invention.

FIG. 8 illustrates view of the BPA-free nozzle tip attached, in accordance with a preferred embodiment of the invention.

FIG. 9 illustrates a flowchart of the method for performing enema and vaginal wash hygiene procedures using the hygiene attachment kit, in accordance with a preferred embodiment of the invention.

### DETAILED DESCRIPTION OF DRAWINGS

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure.

In the following description, numerous specific details are set forth in order to provide a thorough understanding of embodiments of the present invention. It will be apparent to one skilled in the art that embodiments of the present invention may be practiced without some of these specific details.

Those skilled in the art will understand that when an element or part in the drawings is referred to as being “on” (or “connected” to or “coupled” to or “attached” to) another element, it can be directly on (or attached to) the other element or intervening elements may also be present. Furthermore, relative terms such as “inner”, “outer”, “upper”, “above”, “lower”, “beneath”, and “below”, and similar terms, may be used herein to describe a relationship of one element to another element. It is understood that these terms are intended to encompass different orientations of the device in addition to the orientation depicted in the figures.

A hygiene showerhead and sink faucet attachment kit for enema and vaginal wash procedures is designed to enhance comfort, effectiveness, and safety during cleansing routines. The showerheads and sink faucet attachment kit includes several components aimed at facilitating a seamless and hygienic experience. Firstly, it comprises at least one nozzle tip attachment, strategically crafted to promote both comfort and efficacy in the cleansing process. This showerheads and sink faucet attachment ensures that users can undergo the procedure without discomfort while achieving thorough cleaning.

Moreover, the showerhead and sink faucet attachment kit features a water flow pressure regulator switch valve, allowing users to adjust the flow rate and pressure of the cleaning water according to their preferences and needs. This capability adds a layer of control and customization to the cleansing process, ensuring a tailored experience for each user. To transport the cleaning water from a water source, the kit includes a flexible silicone tube-hose. This hose is specifically designed to be pliable and durable, enabling easy handling and maneuverability during use. Ensuring a secure and leak-proof connection, a segment of the tube-hose connects to the water regulator switch valve. This connection is essential to maintain the integrity of the cleansing procedure and prevent any unwanted leaks or spills.

In addition, a water filtration mesh washer is integrated into the kit to filter out particles or debris from the tube-hose, guaranteeing clean and safe hygiene procedures. This filtration mechanism enhances the overall hygiene standards of the procedure, promoting user safety and comfort. For connecting to a water inlet, the kit includes both male and female PVC threaded coupling connector fittings. These fittings enclose and secure the water filtration washer and a water inlet lock-in washer, providing a reliable and secure connection point.

To ensure compatibility with various water sources, a stretchable universal showerhead and sink faucet water inlet rubber silicone attachment are included. This attachment offers versatility and convenience, allowing users to perform the cleansing procedure with ease regardless of the water source available. Crucially, the water inlet lock-in washer plays a vital role in securing and locking the stretchable universal showerhead and sink faucet water inlet rubber silicone attachment inside the male and female threaded coupling connector fittings. This mechanism guarantees a leak-proof connection, preventing any water leakage during the hygiene procedures and ensuring a hassle-free experience for users. This component ensures compatibility with a wide range of water sources, thereby enhancing the kit's versatility and convenience for users, allowing for seamless integration into various hygiene routines.

The hygiene showerhead and sink faucet attachment kit described includes a variety of BPA-free nozzle tip attachments, each designed with tapered ends and bulbous shapes to cater to different user preferences and anatomical needs. These diverse nozzle tips ensure versatility and comfort during cleansing procedures. Moreover, the kit incorporates a water flow pressure regulator switch valve, allowing users precise control over the intensity of the water flow. This feature enables customization of the cleansing experience, ensuring comfort and effectiveness tailored to individual preferences.

Additionally, various essential components of the kit, such as the nozzle tip attachment, the water flow pressure regulator switch valve, the male bottom PVC threaded coupling connector fitting, the female upper PVC threaded coupling connector fitting, and the water inlet lock-in washer, are constructed from BPA-free food-grade PVC material. This material choice prioritizes safety and hygiene standards, ensuring that users are not exposed to harmful substances during their hygiene procedures. Furthermore, the kit features a flexible silicone tube-hose, specifically designed to transport cleaning water between the nozzle tip, the switch valve, and the water inlet rubber silicone attachment. This hose facilitates a smooth and efficient flow of water, ensuring that users can perform their hygiene routines with ease and comfort while maintaining optimal cleanliness and safety standards.

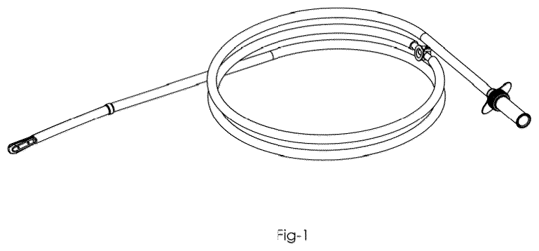

FIG. 1 illustrate an attachment for enema and vaginal wash hygiene, in accordance with a preferred embodiment of the invention.

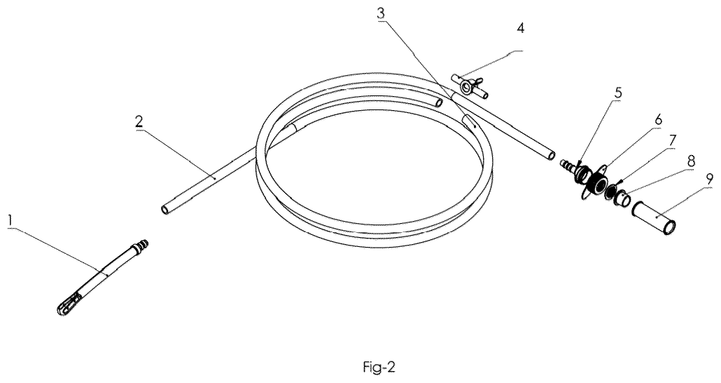

FIG. 2 illustrates various elements of the attachment for enema and vaginal wash hygiene, in accordance with a preferred embodiment of the invention. The individual parts of the attachment and their functionality to providing a comprehensive solution for enema and vaginal wash procedures is explained below:

Nozzle Tip Attachment (1) for the hygiene attachment kit, in accordance with a preferred embodiment of the invention. The hygiene Attachment Kit comes with a selection of nozzle tip attachments, each made from BPA-free materials to ensure safety and hygiene. These nozzle tips are available in various shapes and sizes depending on the model, catering to different user preferences and anatomical needs. For enema procedures, the kit may include nozzle tips with tapered ends or bulbous shapes to facilitate insertion and comfortable use. For vaginal wash hygiene, nozzle tips may be designed with gentle curves or angles to provide optimal cleansing and comfort. These nozzle tip attachments are ergonomically designed to ensure ease of use and effectiveness in achieving thorough cleansing.

Flexible, Medical-grade BPA-Free Silicone Tube-Hose (2): Facilitating the transport of cleaning water between the nozzle tips, switch valve, and water inlet rubber silicone attachment, the Hygiene Attachment Kit features a flexible tube-hose made from medical-grade, BPA-free silicone. This tube-hose is durable, hygienic, and flexible, allowing for easy maneuverability during hygiene procedures. Its smooth surface minimizes the risk of irritation or discomfort during use, ensuring a comfortable experience for the user. The tube-hose is designed to withstand repeated use and can be easily cleaned and sanitized for optimal hygiene maintenance.

Segment of the Tube-Hose Connecting to the Water Regulator Switch Valve (3): This segment of the tube-hose serves as the connection point between the water flow pressure regulator switch valve and the main tube-hose assembly. Made from flexible, medical-grade BPA-free silicone, this segment ensures a secure and leak-proof connection, maintaining optimal hygiene and safety standards during use. Its flexibility allows for easy installation and adjustment, ensuring a seamless integration with the rest of the attachment kit components.

Water Flow Pressure Regulator Switch Valve (4): The Hygiene Attachment Kit is equipped with a water flow pressure regulator switch valve, allowing users to adjust the flow rate and pressure of the cleaning water according to their preferences and comfort levels. This switch valve provides precise control over the intensity of the water flow, ensuring a customized and comfortable experience for the user. Whether for gentle cleansing or more thorough rinsing, the water flow pressure regulator switch valve allows users to tailor the hygiene procedure to their individual needs.

Male Bottom PVC Threaded Coupling Connector Fitting (5): Enclosing and securing the water filtration washer and water inlet lock-in washer, the male bottom PVC threaded coupling connector fitting is an essential component of the Hygiene Attachment Kit. This fitting is designed to connect the main tube-hose assembly to the water source, such as a showerhead or sink faucet. Its threaded design ensures a secure and tight seal, preventing leaks and ensuring a continuous supply of cleaning water during hygiene procedures. The male bottom PVC threaded coupling connector fitting is made from BPA-free materials, ensuring safety and compatibility with food-grade standards.

Female Upper PVC Threaded Coupling Connector Fitting (6): Similar to the male bottom PVC threaded coupling connector fitting, the female upper PVC threaded coupling connector fitting encloses and secures the water filtration washer and water inlet lock-in washer. This fitting serves as the connection point between the main tube-hose assembly and the water source, providing a secure and leak-proof seal. Its threaded design allows for easy installation and removal, ensuring a hassle-free experience for the user. Like all components of the Hygiene Attachment Kit, the female upper PVC threaded coupling connector fitting is made from BPA-free materials to ensure safety and hygiene.

Water Filtration Mesh Washer (7): The water filtration mesh washer is a crucial component of the Hygiene Attachment Kit, responsible for filtering out particles or debris from the water tube-hose line. Made from durable and hygienic materials, this mesh washer effectively removes impurities from the cleaning water, ensuring a clean and safe hygiene procedure. Its fine mesh design allows for efficient filtration without impeding the flow of water, maintaining optimal performance and reliability. The water filtration mesh washer is designed to be easily removable and cleanable, allowing for hassle-free maintenance and upkeep of the attachment kit.

Water Inlet Lock-in Washer (8): The water inlet lock-in washer plays a vital role in securing and locking the stretchable universal showerhead and sink faucet water inlet rubber silicone attachment inside the male and female threaded coupling connector fittings. This washer ensures a tight and leak-proof seal, preventing water from escaping and maintaining optimal pressure during hygiene procedures. Its durable construction and secure locking mechanism provide peace of mind to users, ensuring a reliable and hassle-free experience. The water inlet lock-in washer is made from BPA-free materials, ensuring safety and compatibility with food-grade standards.

Stretchable Universal Showerhead and Sink Faucet Water Inlet Rubber Silicone Attachment (9): Designed for versatility and compatibility, the stretchable universal showerhead and sink faucet water inlet rubber silicone attachment allows the Hygiene Attachment Kit to connect seamlessly to a wide range of water sources. This attachment features a flexible and stretchable design, ensuring compatibility with various showerheads and sink faucet. Its rubber silicone construction provides a secure and leak-proof seal, preventing water from escaping and ensuring a continuous supply of cleaning water during hygiene procedures. The stretchable universal showerhead and sink faucet water inlet rubber silicone attachment is made from BPA-free materials, ensuring safety and hygiene compliance.

All parts of the Hygiene Attachment Kit are crafted from BPA-free and food-grade materials, prioritizing safety, hygiene, and user satisfaction. Each component is meticulously designed and engineered to provide a convenient, effective, and comfortable solution for enema and vaginal wash hygiene practices. With its innovative design, versatile functionality, and commitment to quality, the Hygiene Attachment Kit sets a new standard in personal hygiene technology, offering users a reliable and hygienic solution for their hygiene needs.

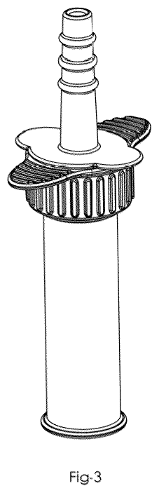

FIG. 3 illustrates connection and filtration part of the attachment for enema and vaginal wash hygiene, in accordance with a preferred embodiment of the invention. It comprises essential components numbered 5 to 9 within the hygiene attachment kit, each serving a distinct role in ensuring efficient water supply, secure connections, and effective filtration during hygiene procedures

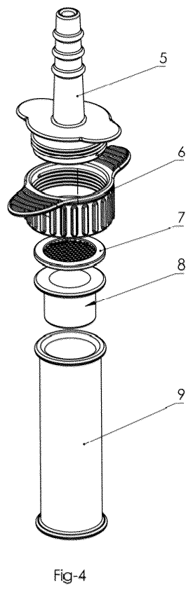

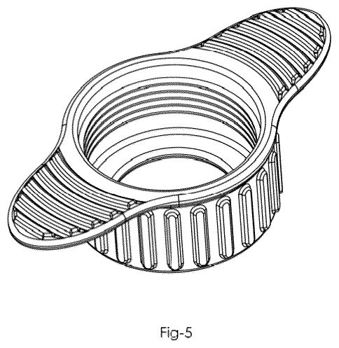

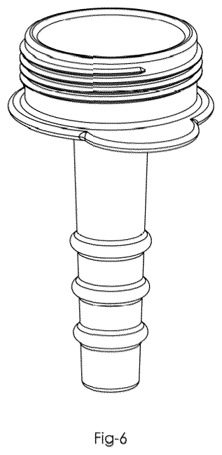

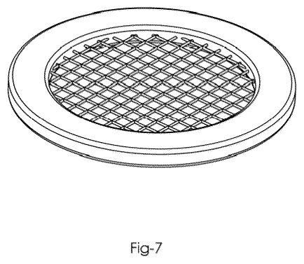

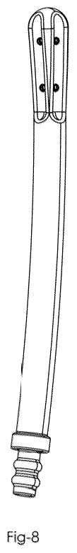

FIG. 4-8 illustrates in details all parts of the connection and filtration part of the attachment for enema and vaginal wash hygiene, in accordance with a preferred embodiment of the invention. It consists of the male bottom PVC threaded coupling connector fitting (5). This fitting encloses and secures critical elements such as the water filtration washer and water inlet lock-in washer, providing a stable and leak-proof connection to the water source. Further, the female upper PVC threaded coupling connector fitting (6), facilitates a leak-proof connection to the water source but also ensures the secure enclosure of essential washers. The Water Filtration Mesh Washer (7) plays a vital role in maintaining hygiene standards by filtering out particles or debris from the water, thereby ensuring the cleanliness and safety of the hygiene procedure. Water Inlet Lock-in Washer (8) is instrumental in securing and locking the stretchable universal showerhead and sink faucet water inlet rubber silicone attachment within the male and female threaded coupling connector fittings. This prevents water leakage and helps maintain optimal pressure during the procedure. The Stretchable Universal Water Inlet Attachment (9) provides versatility by enabling compatibility with various water sources, such as showerheads or sink faucet. This attachment enhances convenience and ease of use for users, ensuring a seamless and adaptable experience during hygiene procedures. Together, these components form a cohesive unit within the hygiene attachment kit, ensuring reliability, safety, and effectiveness throughout the enema and vaginal wash procedures.

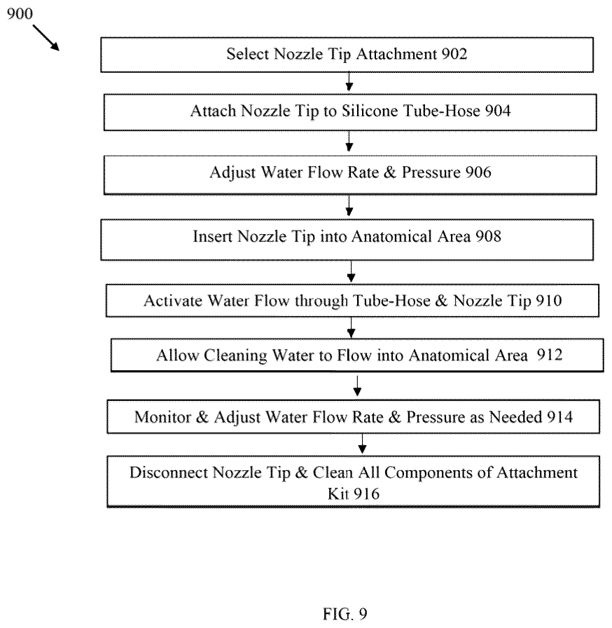

FIG. 9 illustrates a flowchart of the method for performing enema and vaginal wash hygiene procedures using the hygiene attachment kit, in accordance with a preferred embodiment of the invention. The explanation of each step in the method (900) for performing enema and vaginal wash hygiene procedures using a hygiene attachment kit is as mentioned below:

At Step 902, Select Nozzle Tip Attachment: In this step, the user selects a suitable nozzle tip attachment from a variety of options provided by the hygiene attachment kit. These nozzle tip attachments come in various shapes and sizes to accommodate different user preferences and anatomical needs. For example, some attachments may have tapered ends or bulbous shapes for easier insertion and comfortable use. The user chooses the nozzle tip attachment that best suits their requirements for the cleansing procedure.

At Step 904, Attach Nozzle Tip to Silicone Tube-Hose: Once the nozzle tip attachment is selected, it is securely attached to the flexible silicone tube-hose of the hygiene attachment kit. This attachment ensures a proper connection between the nozzle tip and the tube-hose, preventing any leaks or disconnections during the cleansing procedure. The attachment process is straightforward and ensures that the nozzle tip remains firmly in place throughout the procedure.

At Step 906, Adjust Water Flow Rate & Pressure: In this step, the user adjusts the water flow rate and pressure using the pressure regulator valve provided in the hygiene attachment kit. The pressure regulator valve allows users to customize the intensity of the water flow according to their preferences and comfort levels. By adjusting the valve, users can increase or decrease the flow rate and pressure of the cleaning water to ensure a comfortable and effective cleansing experience.

At Step 908, Insert Nozzle Tip into Anatomical Area: With the attachment of the nozzle tip to the tube-hose and adjustment of water flow parameters completed, the user inserts the attached nozzle tip into the desired anatomical area for cleansing. It's essential to ensure proper positioning of the nozzle tip to target the specific area requiring cleansing, whether for enema or vaginal wash procedures. Proper insertion helps to maximize the effectiveness of the cleansing process.

At Step 910, Activate Water Flow through Tube-Hose & Nozzle Tip: Once the nozzle tip is inserted into the anatomical area, the user activates the water flow through the tube-hose and nozzle tip attachment by opening the water source. This could be a showerhead or a sink faucet, depending on the setup. Activating the water flow initiates, the cleansing process, with water flowing through the tube-hose and nozzle tip into the targeted anatomical area for thorough cleansing.

At Step 912, Allow Cleaning Water to Flow into Anatomical Area: As the water flows through the tube-hose and nozzle tip attachment, it enters the anatomical area, effectively performing the enema or vaginal wash procedure. The user allows the cleaning water to flow into the area, ensuring thorough cleansing and removal of impurities or debris. It's essential to maintain a steady and consistent flow of water to achieve optimal results.

At Step 914, Monitor & Adjust Water Flow Rate & Pressure as Needed: Throughout the cleansing procedure, the user monitors the water flow and adjusts the flow rate and pressure as needed to maintain comfort and effectiveness. By periodically checking the water flow and pressure, users can ensure that the cleansing process remains comfortable and that adequate cleansing is achieved. Adjustments can be made using the pressure regulator valve to tailor the experience to individual preferences.

At Step 916, Disconnect Nozzle Tip & Clean All Components of Attachment Kit: After completing the cleansing procedure, the user disconnects the nozzle tip attachment from the tube-hose. All components of the hygiene attachment kit, including the nozzle tip, tube-hose, and any other attachments, are thoroughly cleaned to maintain hygiene standards. Proper cleaning ensures that the attachment kit remains safe and hygienic for future use, preventing the buildup of bacteria or contaminants. This step is essential for preserving the effectiveness and longevity of the hygiene attachment kit.

The invention of the hygiene attachment kit for enema and vaginal wash procedures offers several advantages:

Versatility: The attachment kit is compatible with various water sources such as showerheads and sink faucet, providing users with flexibility in different environments.

Customization: Users can select from different nozzle tip attachments and adjust water flow rate and pressure according to their preferences and comfort levels, ensuring a personalized cleansing experience.

Hygiene: The kit is made from medical-grade, BPA-free materials, promoting hygiene and safety during use. Components can be easily cleaned after each use to maintain cleanliness standards.

Comfort: The nozzle tip attachments are designed with features like tapered ends or bulbous shapes to accommodate different anatomical needs, enhancing user comfort during cleansing procedures.

Ease of Use: The attachment kit is user-friendly and straightforward, allowing users to perform enema and vaginal wash procedures conveniently and effectively.

Efficiency: The integrated water filtration mesh washer helps to filter out particles or debris from the water, ensuring clean and safe hygiene procedures.

Adaptability: With a stretchable universal water inlet rubber silicone attachment, the kit can be easily connected to different water sources, making it suitable for use in various settings.

While the subject invention is described and illustrated with respect to certain preferred and alternative embodiments, it should be understood that various modifications can be made to those embodiments without departing from the subject invention, the scope of which is defined in the following claims.

1. A hygiene showerhead and sink faucet attachment kit for enema and vaginal wash procedures, comprising:
at least one nozzle tip attachment to facilitate comfortable and effective cleansing;
a water flow pressure regulator switch valve, allowing users to adjust the flow rate and pressure of the cleaning water;
a flexible silicone tube-hose designed to transport cleaning water from a water source;
a segment of the tube-hose connecting to the water regulator switch valve to ensure a secure and leak-proof connection;
a water filtration mesh washer to filter out particles or debris from the tube-hose ensuring clean and safe hygiene procedures;
a male bottom PVC threaded coupling connector fitting, enclosing and securing the water filtration washer and a water inlet lock-in washer, providing a secure connection to a water inlet;
a female upper PVC threaded coupling connector fitting, enclosing and securing the water filtration washer and the water inlet lock-in washer, facilitating a leak-proof connection to the water inlet;
a stretchable universal showerhead and sink faucet water inlet rubber silicone attachment, designed to provide compatibility with various water sources, ensuring convenience and ease of use for users;
wherein the water inlet lock-in washer secures and locks the stretchable universal showerhead and sink faucet water inlet rubber silicone attachment inside the male and female threaded coupling connector fittings, ensuring a leak-proof connection and preventing water leakage during hygiene procedures.

2. The hygiene showerhead and sink faucet attachment kit of claim 1, wherein the different BPA-free nozzle tip attachments include tapered ends and bulbous shapes to accommodate different user preferences and anatomical needs.

3. The hygiene showerhead and sink faucet attachment kit of claim 1, wherein the water flow pressure regulator switch valve allows for precise control over the intensity of the water flow, ensuring a customized and comfortable hygiene experience for users.

4. The hygiene showerhead and sink faucet attachment kit of claim 1, wherein the water filtration mesh washer effectively removes impurities from the cleaning water, ensuring a clean and safe hygiene procedure.

5. The hygiene showerhead and sink faucet attachment kit of claim 1, wherein the stretchable universal showerhead and sink faucet water inlet rubber silicone attachment ensures compatibility with a wide range of water sources, enhancing versatility and convenience for users.

6. The hygiene showerhead and sink faucet attachment kit of claim 1, wherein the nozzle tip attachment, the water flow pressure regulator switch valve, the male bottom PVC threaded coupling connector fitting, the female upper PVC threaded coupling connector fitting and the water inlet lock-in washer are made of BPA-free food-grade PVC material.

7. The hygiene showerhead and sink faucet attachment kit of claim 1, wherein the flexible silicone tube-hose is designed to transport cleaning water between the nozzle tip, the switch valve, and the water inlet rubber silicone attachment.

8. A method for performing enema and vaginal wash hygiene procedures using a hygiene showerhead and sink faucet attachment kit, comprising the steps of:
selecting a nozzle tip attachment from a plurality of nozzle tip attachments, each available in various shapes and sizes to facilitate comfortable and effective cleansing;
attaching the selected nozzle tip attachment to a flexible, medical-grade BPA-free silicone tube-hose of the hygiene attachment kit, ensuring a secure connection;
adjusting water flow rate and pressure using a water flow pressure regulator switch valve of the hygiene attachment kit, according to user preferences;
inserting the attached nozzle tip into the desired anatomical area for cleansing ensuring proper positioning;
activating the water flow through the tube-hose and nozzle tip attachment by opening the water source, such as a showerhead or sink faucet;
allowing the cleaning water to flow through the nozzle tip attachment and into the anatomical area, effectively performing the enema or vaginal wash procedure;
monitoring the water flow and adjusting the water flow rate and pressure as needed to maintain comfort and effectiveness throughout the procedure;
disconnecting the nozzle tip attachment from the tube-hose and cleaning all components of the hygiene attachment kit thoroughly after use to maintain hygiene standards.

9. The method of claim 8, wherein the nozzle tip attachment includes a tapered end or bulbous shape to accommodate different user preferences and anatomical needs.

10. The method of claim 8, wherein the adjustment of water flow rate and pressure is tailored to individual user comfort levels and anatomical requirements ensuring a personalized and effective cleansing experience.
